(19) United States
(12) Patent Application Publication
(10) Pub. No.: US 2024/0277282 A1
(43) Pub. Date: Aug. 22, 2024

(54) AUTOMATED DETECTION OF COGNITIVE CONDITIONS

(71) Applicant: ROWAN UNIVERSITY, Glassboro, NJ (US)

(72) Inventors: Ganesh BALIGA, Glassboro, NJ (US); David J. LIBON, Glassboro, NJ (US)

(73) Assignee: ROWAN UNIVERSITY, Glassboro, NJ (US)

(21) Appl. No.: 18/443,973

(22) Filed: Feb. 16, 2024

Related U.S. Application Data
(60) Provisional application No. 63/446,548, filed on Feb. 17, 2023.

(51) Int. Cl. A61B 5/00; G16H 50/20

(52) U.S. Cl. CPC A61B 5/4088; A61B 5/748; G16H 50/20

(57) ABSTRACT

One aspect of the present disclosure provides for a computer-implemented method for automated detection of cognitive conditions as described herein (e.g., to detect emergent neurocognitive decline consistent with mild cognitive impairment (MCI) and Alzheimer's Disease and Related Dementias (ADRD)).

### CROSS-REFERENCE TO RELATED APPLICATIONS

The present application claims priority under 35 U.S.C. § 119(c) to U.S. Provisional Patent Application No. 63/446,548, filed Feb. 17, 2023, which is hereby incorporated herein by reference in its entirety.

### BACKGROUND

Alzheimer's Disease and Related Dementias (ADRD) are major public health problems, posing tremendous stress to global healthcare delivery systems. Neurological conditions such as dementia (including Alzheimer's disease and related dementia syndromes) and prodromal neurological syndromes (such as mild cognitive impairment (MCI) conditions) are known to cause impairment in neuropsychological abilities (including executive control, episodic memory, language, thinking, and judgment) that are greater than typical, age-related declines. However, conditions related to MCI may be difficult to identify or diagnose, particularly when patients are comparatively young and medically healthy.

Neuropsychological assessments designed to identify neurodegenerative diseases such as dementia rely on pen-and-paper tests administered by a trained medical practitioner such as a neuropsychologist. Neuropsychological assessments in the art are often time-consuming, expensive to administer, not culturally neutral, and/or not easy to translate to other languages.

### SUMMARY

One aspect of the present disclosure provides for a computer-implemented method for automated detection of cognitive conditions as described herein (e.g., to detect emergent neurocognitive decline consistent with mild cognitive impairment (MCI) and Alzheimer's

Disease and Related Dementias (ADRD)). The computer-implemented method includes: (a) providing a first display on an electronic screen, the first display including: a first plurality of characters, the first plurality of characters including a designated first target character; and a first instruction to a user to select the first target character; (b) receiving a first input from the user; (c) providing a second display on the electronic screen, the second display including: a second plurality of characters, the second plurality of characters including a designated second target character; and a second instruction to the user to select the second target character; (d) receiving a second input from the user; (e) providing a third display on the electronic screen, the third display including: a third plurality of characters, the third plurality of characters including a designated third target character and a designated fourth target character; and a third instruction to the user to select the third target character and the fourth target character in alternation; (f) receiving a third input from the user; and (g) analyzing the first input, the second input, and the third input to determine a condition of the user.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

The invention is best understood from the following detailed description when read in connection with the accompanying drawing. It is emphasized that, according to common practice, the various features of the drawing are not to scale. On the contrary, the dimensions of the various features are arbitrarily deformed or reduced for clarity. Included in the drawing are the following figures:

FIGS. 1A-1C illustrate various displays of a device in accordance with certain exemplary embodiments of the present disclosure;

FIG. 2 illustrates a system for automated detection of cognitive conditions, according to an embodiment of the present disclosure; and

FIG. 3 illustrates a flow diagram for implementing a method according to an embodiment of the present disclosure.

### DETAILED DESCRIPTION OF THE INVENTION

The present disclosure describes a system and method for identifying or for automated detection of neurologic conditions such as, but not limited to, mild cognitive impairment (MCI) and Alzheimer's Disease and Related Dementias (ADRD).

Neuropsychological assessments designed to MCI and ADRD often rely on pen-and-paper tests administered by a trained medical practitioner (e.g., a neuropsychologist). Digitally administering such assessments can provide traditional test scoring metrics as well as many previously unobtainable time-based parameters, such as inter-response latency. Recent research has demonstrated that these digital assessments are able to identify neurocognitive impairments earlier that their traditional counterparts.

Certain embodiments of the present disclosure provide an effective assessment of executive abilities, a construct highly associated with MCI and ADRD and could be an effective test to screen for neurocognitive impairment (e.g., in the context of elevated cardiovascular risk in an ambulatory primary care environment). Certain embodiments of the present disclosure can be a neuropsychological test administered in the office setting, while gathering additional information provided by a patient's motor responses.

Certain embodiments of the present disclosure can implement a cancellation test, sometimes referred to herein as the “Rowan Digital Cancellation Test” (RDCT). Such an embodiment can include three subtests, where each subtest can be implemented via a screen that contains characters. A “character” can be a letter or a symbol. The first display on a screen can contain only letters; the second display on the screen can contain only symbols; and the third display on the screen can contain both letters and symbols. Each display can also identify target character(s) (or a set of target characters), where the target character(s) are illustrated or displayed at the top of a display on the screen. A patient can be instructed to circle the characters within the screen's target set in alternation. Certain embodiments of the present disclosure can be implemented on an app (e.g., Apple IOS app) running on a tablet computer (e.g., an iPad). The patient can circle characters on the display using a stylus (e.g., an Apple Pencil) or their finger(s).

Certain embodiments of the present disclosure can also be implemented on other computing devices that have a display and the ability to receive patient input (e.g., from a modern digitized pen). Certain embodiments of the present disclosure support the extraction of digital measures such as the time to circle each character and the latency between characters circled. Research conducted in connection with the present disclosure has demonstrated the added value provided by these digital measures in identifying early emergence of cognitive impairment. Latencies can correlate with certain conditions (e.g., cognitive decline, cognitive impairment, mild cognitive impairment, physiological deficiencies, patient cardiovascular risk, and other conditions).

Certain embodiments of the present disclosure can be described as a test designed to measure executive abilities. Patients can be assessed using the standard Montreal Cognitive Assessment (MoCA) test which provides neuropsychological indices measuring executive, language, and memory abilities.

Certain embodiments of the present disclosure can provide a means to screen for dementia in primary care, specialty care, and other health care venues.

Certain embodiments of the present disclosure can provide certain advantages over related technologies and methods. For example, certain embodiments of the present disclosure can be efficiently implemented, inexpensive to administer, culturally neutral, and/or easy to translate to other languages. Certain embodiments of the present disclosure can provide further advantages over related technologies and methods. For example, certain embodiments of the present disclosure can: (1) be administered electronically in the PCP or internal medicine environment requiring a minimum of technological skill; (2) demonstrate sensitivity in identifying emergent illness; (3) be repeatable or can be re-administered as clinically indicated; (4) be easily translated into any language; (5) be culturally neutral; (6) be reimbursable and can be integrated into existing CPT codes; and (7) be affordably and efficiently implemented. Certain tests of the present disclosure can be easily administered and scored (unlike some digital tests) because there can be minimal (or no) post processing of data.

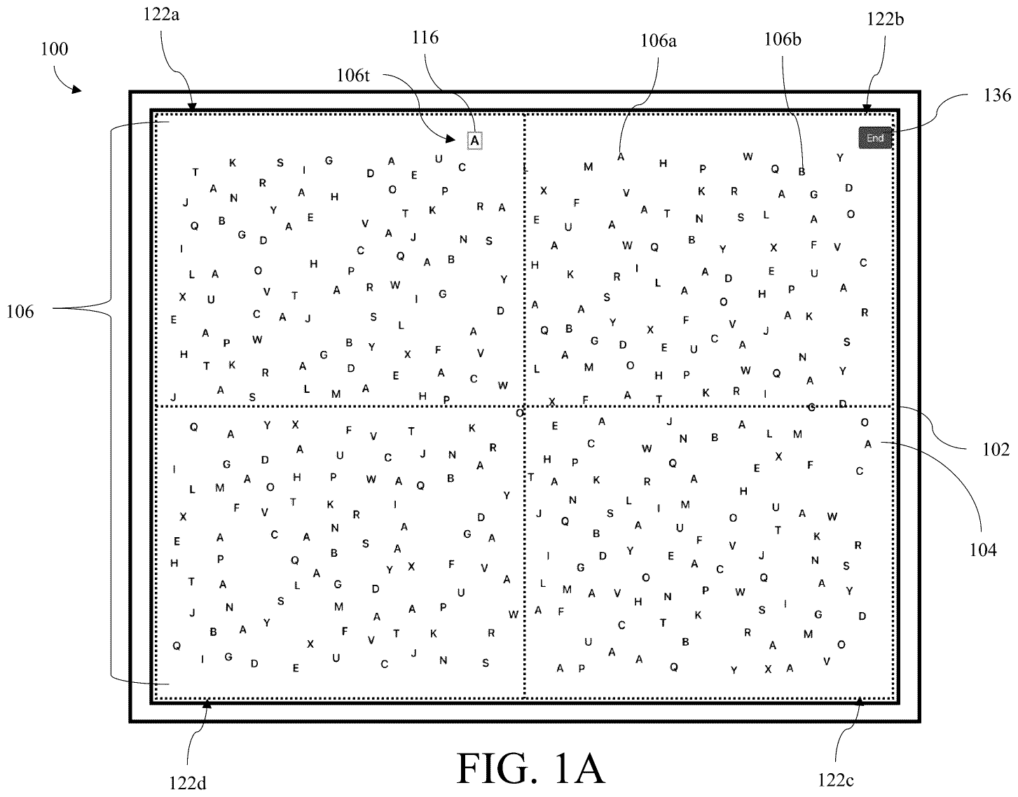

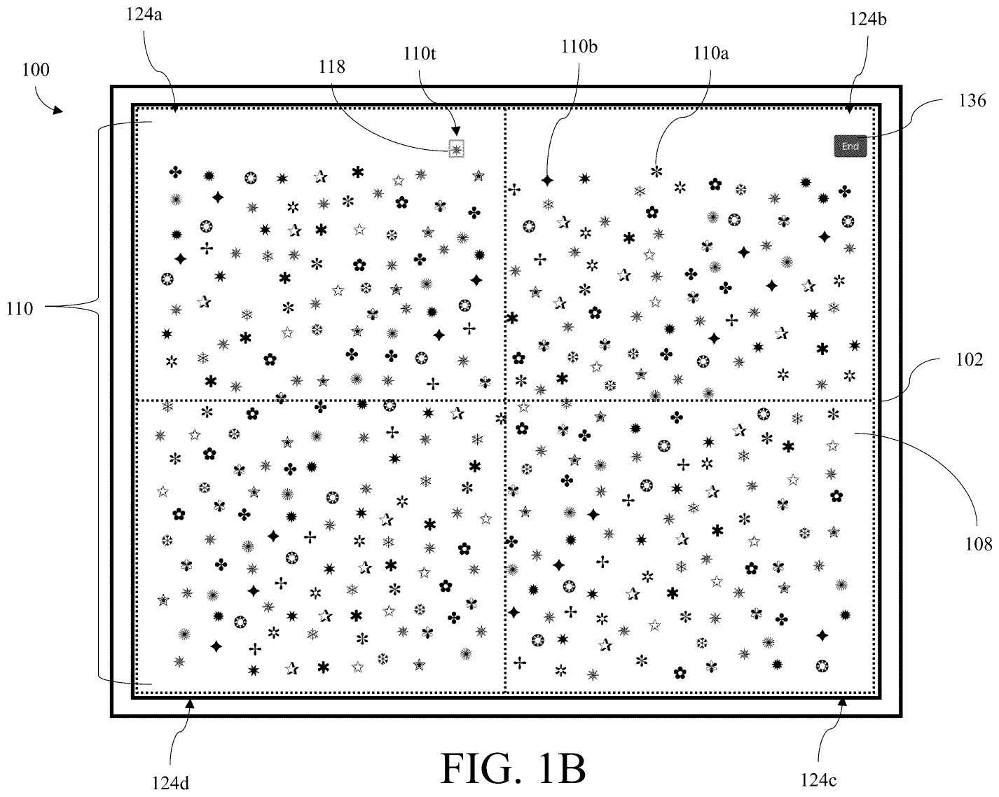

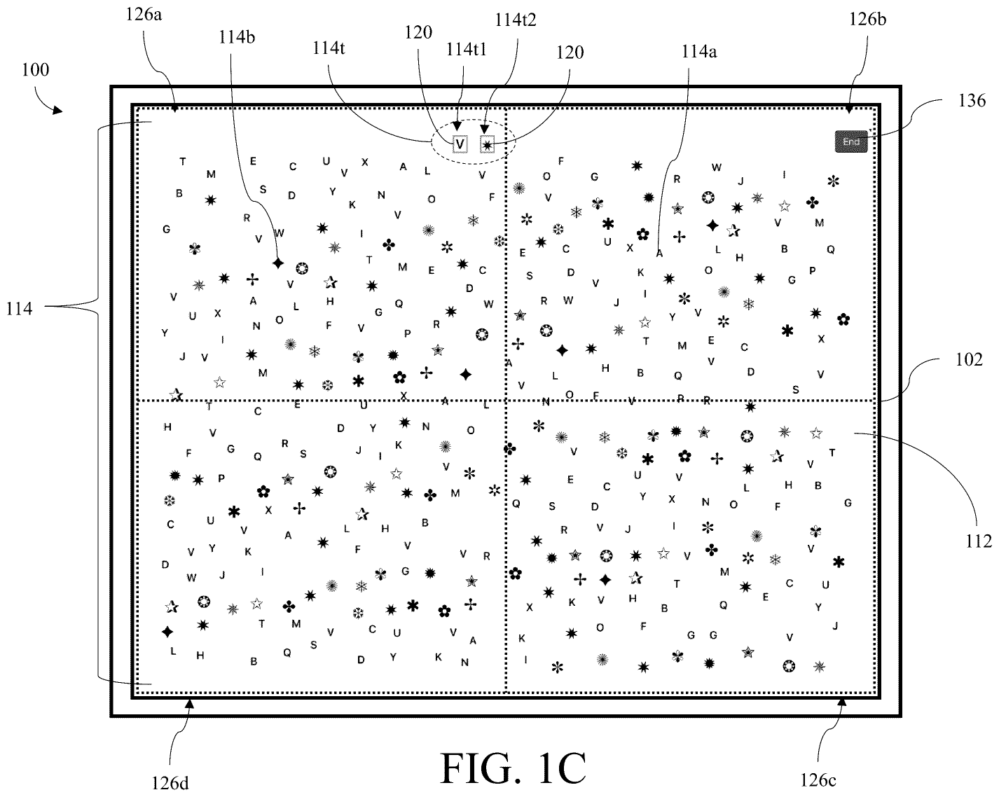

Referring now to the drawings, FIGS. 1A-1C illustrate a system 100 configured and adapted to implement certain methods of the present disclosure. Various tests that are implemented to determine neurological or medical impairment may be utilized by the system 100. System 100 (e.g., a tablet computer) can include an electronic screen 102 configured and adapted to implement a display. The tablet computer is configured to collect certain information, measurements, and/or data relating to the patient's interactions with the screen 102 during the various tests. This information, measurements, and/or data can then be used to assess underlying cognitive abilities.

Referring specifically to FIG. 1A, a first display 104 is provided on electronic screen 102. The first display 104 includes/displays a first plurality of characters 106. In some embodiments, the first plurality of characters include, but are not limited to, letters (e.g., character 106a being the letter “A”; character 106b being the letter “B”, and so forth). The patient is instructed how to interact with one or more designated target characters 106t, which may be any of the first plurality of characters 106 (e.g., the target character 106t is shown as the letter “A” in FIG. 1A). For example, a patient can be instructed to: interact with (e.g., circle) the target character 106t (e.g., “A”) in certain sections (e.g., quadrant 122a) of a plurality of sections (e.g., quadrants 122a, 122b, 122c, or 122d); interact with a certain number of instances of the target character 106t (e.g., circling “A” one time, two times, three times, and so forth); or a combination thereof (e.g., circling “A” three times in quadrant 122a and/or then circling “A” three times in quadrant 122b, and so forth). The instructions can be provided to the patient in any suitable manner. For example, in some embodiments, a first instruction to select a target character 106t is provided to the patient on the first display 104 (e.g., in an instruction box 116, as text in a section of the first display 104, or any other suitable manner visible on the screen 102). Additionally or alternatively, in some embodiments, the instructions are communicated aurally (e.g., through a speaker, verbally from a human, and so forth) before or during implementation of a test of the present disclosure. After instructions have been communicated to the patient, the patient interacts with the first display 104 of electronic screen 102 and the tablet computer collects information, measurements, and/or data relating to the patient's interactions (e.g., patent's input to system 100). Once a certain event has been achieved (e.g., successful completion of the task according to instructions; a predefined time period elapsing; and so forth), a second display can be provided.

Referring now to FIG. 1B, a second display 108 is provided on electronic screen 102. The second display 108 includes/displays a second plurality of characters 110. In some embodiments, the second plurality of characters 110 includes symbols (e.g., character 110a being the symbol “ [symbol] ”; character 110b being the symbol “ [symbol] ”; and so forth). The patient is instructed how to interact with one or more designated target characters 110t, which may be any of the second plurality of characters 110 (e.g., the target character 110t is shown as the symbol “ [symbol] ” in FIG. 1B). For example, a patient can be instructed to: interact with (e.g., circle) the target character 110t (e.g., “ [symbol] ”) in certain sections (e.g., quadrant 124a) of a plurality of sections (e.g., quadrants 124a, 124b, 124c, or 124d); interact with a certain number of instances of the target character 110t (e.g., circling “ [symbol] ” one time, two times, three times, and so forth); or a combination thereof (e.g., circling “ [symbol] ” three times in quadrant 124a and/or then circling “ [symbol] ” three times in quadrant 124b, and so forth). The instructions can be provided to the patient in any suitable manner. For example, in some embodiments, a second instruction to select a target character 110t is provided to the patient on the second display 108 (e.g., in an instruction box 118, as text in a section of the second display, or any other suitable manner visible on the screen 102). Additionally or alternatively, in some embodiments, the instructions are communicated aurally (e.g., through a speaker, verbally from a human, and so forth) before or during implementation of a test of the present disclosure. After instructions have been communicated to the patient, the patient interacts with the second display 108 of electronic screen 102 and the tablet computer collects information, measurements, and/or data relating to the patient's interactions (e.g., patent's input to system 100). Once a certain event has been achieved (e.g., successful completion of the task according to instructions; a predefined time period elapsing; and so forth), a third display can be provided.

Referring now to FIG. 1C, a third display 112 is provided on electronic screen 102. The third display 112 includes/displays a third plurality of characters 114. In some embodiments, the third plurality of characters 114 includes a combination of letters and symbols (e.g., character 114a being the letter “A”; character 114b being the symbol “ [symbol] ”; and so forth). The patient is instructed how to interact with one or more designated target characters 114t, which may be any of the third plurality of characters 114 (e.g., the target characters 114t1 and 114t2 are shown as the letter “V” the symbol “ [symbol] ”, respectively, in FIG. 1C). For example, a patient can be instructed to: interact with (e.g., circle) the target character 114t1 (e.g., “V”) in certain sections (e.g., quadrant 126a) of a plurality of sections (e.g., quadrants 126a, 126b, 126c, or 126d); interact with a certain number of instances of the target character 114t1 (e.g., circling “V” one time, two times, three times, and so forth); or a combination thereof (e.g., circling “V” three times in quadrant 126a and/or then circling “V” three times in quadrant 126b, and so forth). Although described primarily with respect to selecting the target character 114t1 above, as will be appreciated by those skilled in the art, the disclosure is not so limited and may include selecting multiple target characters any order and/or combination (e.g., select target character 114t1 and then target character 114t2 in alternation, in groups, in any order, and so forth). The instructions can be provided to the patient in any suitable manner. For example, in some embodiments, a third instruction to select a target character (e.g., 114t1, 114t2, etc.) is provided to the patient on the third display 112 (e.g., in instruction boxes 120, as text in a section of the third display, or any other suitable manner visible on the screen 102). Additionally or alternatively, in some embodiments, the instructions are communicated aurally (e.g., through a speaker, verbally from a human, and so forth) before or during implementation of a test of the present disclosure. After instructions have been communicated to the patient, the patient interacts with the third display 112 of electronic screen 102 and the tablet computer collects information, measurements, and/or data relating to the patient's interactions (e.g., patent's input to system 100). Once a certain event has been achieved (e.g., successful completion of the task according to instructions; a predefined time period elapsing; an “end” button 136 on display 112 is selected; and so forth), the test can be terminated.

In some embodiments, the tests/displays are administered in ascending degrees of hardness (e.g., letters, then symbols, then letters and symbols). However, as will be appreciated by those skilled in the art, the disclosure is not so limited and includes administering the tests/displays in any suitable order, as well as individually, with only two tests/displays, or in combination with additional tests/displays.

In some embodiments, the information, measurements, and/or data collected from each test/display includes, but is not limited to, one or more of the time when a patient interacts with (e.g., touches, circles, and so forth) each character; the latency or time between each interaction; the accuracy of the interactions (e.g., how many times the patient correctly selects the target character in accordance with the instructions vs. the total number of interactions); the orientation of a patient's tablet pen/stylus (e.g., angle of the pen/stylus with respect to the screen 102, direction of the pen/stylus with respect to the patient, or a combination thereof); the pressure applied to the screen 102 by the patient; other measurements; or a combination thereof. Additionally or alternatively, in some embodiments, the information, measurements, and/or data (sometimes referred to as “Digital Cancellation Outcome Measures”) includes, but is not limited to, (1) correct response (i.e., the number of correct responses, e.g., from 0-64); (2) commissions (i.e., the number of erroneously circled false positive foils); (3) percent drawing (i.e., the percent of the total time to completion actually spent drawing with the stylus); (4) percent not drawing (i.e., the percent of total time to completion not drawing with the stylus); (5) total distance (i.e., the length of an imaginary line that connects all correct responses in the order they were circled); (6) mean inter-response latency (i.e., the average time or latency between circling all correct responses); (7) mean touch (i.e., the average number of oscillations of the stylus as the participant circles correct targets); and/or (8) epoch measures (i.e., all measures listed above are calculated for each 30 second time epoch to assess how behavior changes as a function of total time to completion).

After the test has been terminated, system 100 analyzes the input information from one or more of the displays to determine a condition of the patient. For example, in some embodiments, the system 100 analyses the first input (e.g., the input information from the first display 104), the second input (e.g., the input information from the first display 108), and the third input (e.g., the input information from the first display 112) to determine a condition of the user.

The underlying cognitive abilities that are assessed include: (1) Capacity for Engagement (this ability is assessed by tallying the number of correctly identified hits or target items, where a higher hit score suggests an intact capacity to maintain the mental set necessary for successful test completion); (2) Capacity for Disengagement (this ability is assessed by tallying the number of errors or false positive foils, where a higher score suggests de-railed mental set and poor self-monitoring); (3) Decision Making Ratio (this ability is assessed via the calculation of intra-response latencies, or the time elapsed between correct responses divided by the number of correct hits, where slow intra-response latencies suggest sluggish decision making perhaps due to impaired visual scanning abilities); (4) Response Distance Ratio (this ability is assessed by measuring the length of the line between all correct responses divided by the number of correct hits, where a lower value on this metric means that patients are correctly identifying and circling items that are close to each other, therefore, demonstrating an efficient response set; a higher value on this metric means that patients are scanning randomly about the test page and circling items that are distant from each other); (5) Oscillatory Correct Hit Ratio (the number of discrete pen strokes as a patient circles correct responses, where a low score on this metric suggests faster, more parsimonious motor activity and a high score suggests slower, less parsimonious motor activity); and/or (6) Search quality, which is calculated as ((num of correct responses)/(total test time))*((num of correct responses)/(number of targets)).

Patient groups have been shown to be dissociated based on the number of correct targets. Thus, the compilation of the ratio measures using correct hits in the denominator as described above is a way to normalize these metrics. All of these metrics are available in 30 second time epochs to assess how behavior changes (i.e., improves or declines as a function of total time to completion). The statistical analyses undertaken to investigate these outcome variables include an analysis of co-variance to assess for between group differences, logistic regression analysis to assess the ability of these variables to classify patients into their respective groups, and a linear regression analysis to assess how these outcome variables are related to neuropsychological abilities. In certain applications, the speed or accuracy of the user can be compared against a dataset from a previous test of the user or from a dataset of other patients with certain characteristics (e.g., patients with measured cognitive impairment, “healthy” patients, and so forth). The RDCT appears to be well-tolerated in testing performed on community dwelling participants. Preliminary data generally yielded graded pattern of performance based on test complexity. Without wishing to be bound by theory, it is believed that when brought to scale these tests provide a reliable method to screen for neurocognitive decline among patients with neurodegenerative illness.

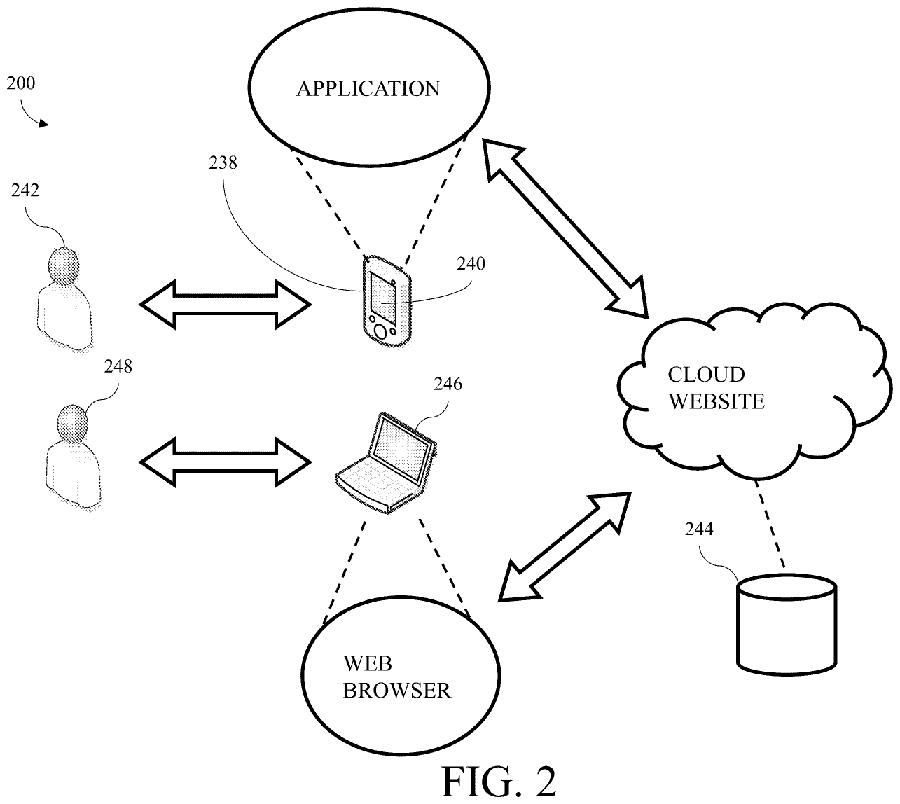

Referring now to FIG. 2, a system 200 configured and adapted for implementing methods (of determining a cognitive condition) of the present disclosure is illustrated. System 200 is illustrated including a device 238 (e.g., a tablet computer) configured and adapted to provide a plurality of displays on a screen 240 of the device 238 (e.g., tablet computer). Screen 240 can be configured and adapted to receive a plurality of inputs from a user 242 (e.g., a subject, a patient, etc.). System 200 (and/or device 238) includes a processing unit configured to analyze the inputs of user 242. Device 238 can implement an application for carrying out certain embodiments of the present disclosure. Device 238 can be configured to interact with a cloud website (hosted by server 244). Device 238 can be configured to interact (directly or indirectly) with a computer 246 operated by a user 248 (e.g., a doctor, a psychologist, or another health care professional). Computer 246 can access results via a web browser in communication with the cloud website.

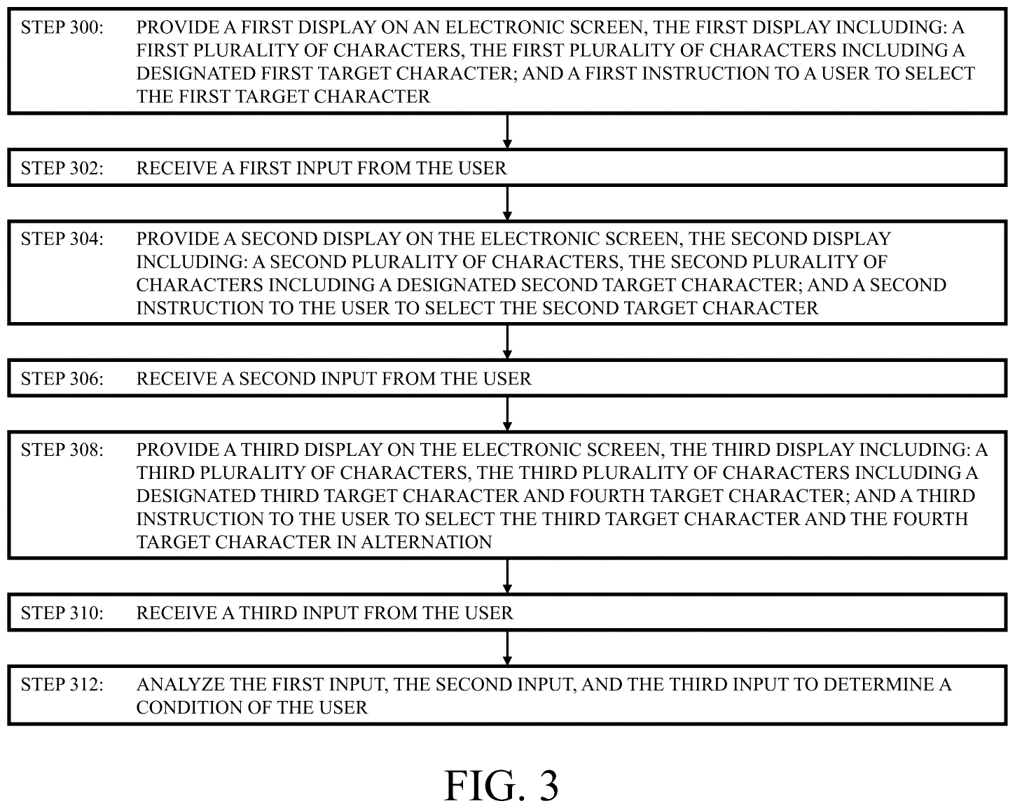

FIG. 3 is a flow diagram in accordance with exemplary embodiments of the present invention. As is understood by those skilled in the art, certain steps included in the flow diagrams may be omitted; certain additional steps may be added; and the order of the steps may be altered from the order illustrated.

FIG. 3 illustrates a method (of determining a cognitive condition). At Step 300, a first display 104 (e.g., first display 104 of FIG. 1A) is provided on an electronic screen (e.g., electronic screen 102 of FIG. 1A), the first display including: a first plurality of characters (e.g., plurality of characters 106 of FIG. 1A), the first plurality of characters including a designated first target character (e.g., first target character 106t of FIG. 1A); and a first instruction to a user to select the first target character.

At Step 302, a first input is received from the user.

At Step 304, a second display (e.g., second display 108 of FIG. 1B) is provided on the electronic screen, the second display including: a second plurality of characters (e.g., plurality of characters 110 of FIG. 1B), the second plurality of characters including a designated second target character (e.g., second target character 110t of FIG. 1B); and a second instruction to the user to select the second target character.

At Step 306, a second input is received from the user.

At Step 308, a third display (e.g., third display 112 of FIG. 1C) is provided on the electronic screen, the third display including: a third plurality of characters (e.g., third plurality of characters 114 of FIG. 1C), the third plurality of characters including a designated third target character and fourth target character (e.g., target character 114t1 and target character 114t2 of FIG. 1C); and a third instruction to the user to select the third target character and the fourth target character in alternation.

At Step 310, a third input is received from the user.

At Step 312, the first input, the second input, and the third input are analyzed to determine a condition of the user (e.g., cognitive impairment).

### Software

The above-described steps can be implemented using standard well-known programming techniques. The novelty of the above-described embodiment lies not in the specific programming techniques but in the use of the steps described to achieve the described results. Software programming code which embodies the present invention is typically stored in permanent storage. In a client/server environment, such software programming code may be stored with storage associated with a server. The software programming code may be embodied on any of a variety of known media for use with a data processing system, such as a diskette, or hard drive, or CD ROM. The code may be distributed on such media or may be distributed to users from the memory or storage of one computer system over a network of some type to other computer systems for use by users of such other systems. The techniques and methods for embodying software program code on physical media and/or distributing software code via networks are well known and will not be further discussed herein.

It will be understood that each element of the illustrations, and combinations of elements in the illustrations, can be implemented by general and/or special purpose hardware-based systems that perform the specified functions or steps, or by combinations of general and/or special-purpose hardware and computer instructions.

These program instructions may be provided to a processor to produce a machine, such that the instructions that execute on the processor create means for implementing the functions specified in the illustrations. The computer program instructions may be executed by a processor to cause a series of operational steps to be performed by the processor to produce a computer-implemented process such that the instructions that execute on the processor provide steps for implementing the functions specified in the illustrations. Accordingly, the figures support combinations of means for performing the specified functions, combinations of steps for performing the specified functions, and program instruction means for performing the specified functions.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

What is claimed is:

1. A computer-implemented method, comprising:
(a) providing a first display on an electronic screen, the first display including:
a first plurality of characters, the first plurality of characters including a designated first target character; and
a first instruction to a user to select the first target character;
(b) receiving a first input from the user;
(c) providing a second display on the electronic screen, the second display including:
a second plurality of characters, the second plurality of characters including a designated second target character; and
a second instruction to the user to select the second target character;
(d) receiving a second input from the user;
(e) providing a third display on the electronic screen, the third display including:
a third plurality of characters, the third plurality of characters including a designated third target character and a designated fourth target character; and
a third instruction to the user to select the third target character and the fourth target character in alternation;
(f) receiving a third input from the user; and
(g) analyzing the first input, the second input, and the third input to determine a condition of the user.

2. The computer-implemented method of claim 1, wherein step (g) includes measuring time to circle each character for each of the first input, the second input, and the third input.

3. The computer-implemented method of claim 2, wherein step (g) further includes measuring latency between characters circled for each of the first input, the second input, and the third input.

4. The computer-implemented method of claim 1, wherein the first plurality of characters are letters.

5. The computer-implemented method of claim 1, wherein the second plurality of characters are symbols.

6. The computer-implemented method of claim 1, wherein the third plurality of characters are a combination of letters and symbols.

7. The computer-implemented method of claim 6, wherein the third target character is a letter, and the fourth target character is a symbol.

8. The computer-implemented method of claim 1, wherein the third instruction to the user includes to select the combination of the third target character and the fourth target character in an alternating sequence.

9. The computer-implemented method of claim 1, wherein the first instruction to the user includes to select the first target character in a first quadrant of the first display.

10. The computer-implemented method of claim 9, wherein the first instruction to the user includes to select the first target character in a plurality of quadrants of the first display.

11. The computer-implemented method of claim 1, wherein the second instruction to the user includes to select the second target character in a first quadrant of the second display.

12. The computer-implemented method of claim 11, wherein the second instruction to the user includes to select the second target character in a plurality of quadrants of the second display.

13. The computer-implemented method of claim 1, wherein the third instruction to the user includes to select the combination of the third target character and the fourth target character in a first quadrant of the third display.

14. The computer-implemented method of claim 13, wherein the third instruction to the user includes to select the third target character and the fourth target character in a plurality of quadrants of the third display.

15. The computer-implemented method of claim 1, wherein the user selects any of the target characters by circling the target characters.

16. A device configured and adapted to implement the method of claim 1 comprising:
a tablet computer configured and adapted to provide a plurality of displays on a screen of the tablet computer; wherein the screen is configured and adapted to receive a plurality of inputs from a user; and
a processing unit configured to analyze the inputs of the user.
